(19) United States
(12) Patent Application Publication
(10) Pub. No.: US 2016/0144033 A1
(43) Pub. Date: May 26, 2016

(54) CONCENTRATED ACETAMINOPHEN SOLUTION

(71) Applicant: Emphascience, Inc., Pittsford, NY (US)

(72) Inventors: Rebanta Bandyopadhyay, Webster, NY (US); Susen Bandyopadhyay, Webster, NY (US); Raman Kapur, Princeton, NJ (US)

(21) Appl. No.: 14/949,008

(22) Filed: Nov. 23, 2015

Related U.S. Application Data
(60) Provisional application No. 62/082,920, filed on Nov. 21, 2014.

(51) Int. Cl. A61K 47/10; A61K 9/08; A61K 31/167

(52) U.S. Cl. CPC A61K 47/10; A61K 31/167; A61K 9/08

(57) ABSTRACT

A concentrated acetaminophen solution is provided that is dissolved in polyethylene glycol (PEG) with trace water. The acetaminophen is present in the PEG at a concentration of at least 200 mg per mL. The composition is useful to deliver or safely store concentrated doses of acetaminophen without degradation of the acetaminophen.

### CROSS-REFERENCE TO RELATED APPLICATIONS

This application claims priority to and is a non-provisional of U.S. Patent Application Ser. No. 62/082,920 (filed Nov. 21, 2014), the entirety of which is incorporated herein by reference.

### BACKGROUND OF THE INVENTION

Many Active Pharmaceutical Ingredients (APIs) are administered in a solution phase for quicker availability at the site of action and at adequate concentration. Accordingly, solution phases are injected when swift action is necessary or the active substance cannot be adequately absorbed by other routes where solid phases are administered, such as by oral ingestion. Even for oral administration, solution compositions are sometimes utilized and can enhance the absorption of the active ingredient into the systemic circulation. This is useful when dissolution of the solid phase in the stomach and the intestines is slow and unpredictable. Yet, compared to solid crystalline phases, challenges involving the designing of solution dosage forms include lack of sufficient solubility of the active in pharmaceutically acceptable solvent media and increased chemical instability.

Acetaminophen is approved to be administered by intravenous infusion to manage mild to moderate pain, moderate to severe pain with adjunctive opioid analgesics and for the reduction of fever. Marketed injection products of acetaminophen require the addition of antioxidants and chelators and the reduction in dissolved oxygen in the medium to retain their chemical stability. For example, an acetaminophen injection such that those sold under the brand name, OFIRMEV®, is stabilized by the addition of cysteine hydrochloride as an antioxidant, dibasic sodium phosphate as a buffer to adjust the pH to approximately 5.5 and by manufacturing and packaging the product under an inert gas atmosphere to reduce the dissolved oxygen content to less than 2 parts per million (ppm). To be administered directly as an infusion, OFIRMEV® also contains a tonicity adjuster, mannitol, which can also act as a metal chelator under certain conditions. Aside from the risks of higher chemical degradation at elevated temperatures, the poor solubility of acetaminophen in water can cause its precipitation from OFIRMEV® at temperatures below 15° C. Care needs to be taken to avoid exposing this solution to colder temperatures, such as refrigeration, or mixing and co-administering with other products to avoid precipitation of one or more of the poorly soluble actives, such as, diazepam and chlorpromazine hydrochloride. An improved composition that deliverys acetaminophen in a solution is therefore desired.

The discussion above is merely provided for general background information and is not intended to be used as an aid in determining the scope of the claimed subject matter.

### BRIEF DESCRIPTION OF THE INVENTION

A concentrated acetaminophen solution is provided that is dissolved in polyethylene glycol (PEG) with trace water. The acetaminophen is present in the PEG at a concentration of at least 200 mg per mL. The composition is useful to deliver or safely store concentrated doses of acetaminophen without degradation of the acetaminophen. An advantage that may be realized in the practice of some disclosed embodiments of the composition is that an acetaminophen solution is provided for use in medical applications that is more stable and more concentrated than conventional acetaminophen solutions.

In a first embodiment, a composition of matter is provided. The composition of matter comprises a polyethylene glycol in liquid form; an acetaminophen dissolved in the polyethylene glycol, the acetaminophen being present in a concentration of at least 200 mg per mL of the polyethylene glycol; and less than 10% (m/v) water.

In a second embodiment, a composition of matter is provided. The composition of matter consists essentially of a polyethylene glycol in liquid form; an acetaminophen dissolved in the polyethylene glycol, the acetaminophen being present in a concentration of at least 200 mg per mL of the polyethylene glycol; and less than 10% (m/v) water.

In a third embodiment, a composition of matter is provided. The composition of matter consists of a polyethylene glycol in liquid form, the polyethylene glycol having an average molecular weight of about 300 g per mole; an acetaminophen dissolved in the polyethylene glycol, the acetaminophen being present in a concentration of at least 200 mg per mL of the polyethylene glycol; and less than 5% (m/v) water.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### DETAILED DESCRIPTION OF THE INVENTION

The subject matter disclosed herein relates to a stable, water-miscible solution of acetaminophen at a concentration higher than its aqueous solubility. The solution can be heat-sterilized and used to safely deliver the effective dose of acetaminophen by a parenteral route without compromising the quality and sterility of the acetaminophen. The subject matter disclosed herein provides a concentrated, water-miscible solution of acetaminophen with a concentration about 200 mg of acetaminophen per ml of polyethylene glycol (PEG) and without any additional component other than trace amounts of water in the range of 0.1-10% (m/v) and storing the composition in a syringe or a polyethylene bag for subsequent dilution to any concentration for suitable mode of administration by a parenteral route without exposing the contents to the ambient, which might result in the loss of sterility. The concentrated formulation may be exposed to a wide range of temperatures without causing the drug substance to precipitate or undergo chemical degradation so as to compromise its safety and use.

Provided herein is a surprisingly simple concentrated solution formulation of acetaminophen in low molecular weight polyethylene glycol that does not precipitate and is chemically stable for extended period of time without the addition of chelators, antioxidants or a buffer. Many molecular weights of polyethylene glycol (PEG) that are liquid at room temperature can be used. In one embodiment the solution of acetaminophen in PEG 300 is used because relatively high proportions of PEG 300 can be administered by intravenous injection. An acetaminophen in PEG 300 solution offers a balance of viscosity, solubility, and manufacturability of the product without compromising its stability. PEG 300 is also compatible with various polyethylene-type flexible containers and syringe barrels, which can accommodate the maximum dose of acetaminophen in a relatively small volume. In one embodiment, acetaminophen in a concentration in the range of 150-225 mg per ml can be dissolved in pure polyethylene glycol with trace amounts of water. This solution can be stored in a suitable container, such as a prefilled syringe or a flexible, intravenous infusion bag, and sterilized.

The concentrated solution may be packaged in a flexible, polyethylene bag containing more than one chamber and physically separated from a suitable diluent until the time of use, when the two compositions can be mixed together to reduce the acetaminophen concentration and the tonicity of the entire composition allowing for intravenous infusion. In one embodiment, the concentrated solution is contained in a prefilled syringe may be directly injected into the blood vessels of a patient as a bolus injection. In another embodiment, a suitable dose of acetaminophen in the concentrated solution may be mixed with a prepackaged injection medium for delivery by intravenous infusion. In another embodiment, the diluent medium can be prepackaged with the concentrated solution of acetaminophen, but separated into distinct immiscible chambers, which can be filled, sterilized and then mixed at the time of use without microbial contamination. The concentrated solution can either be directly administered or mixed with suitable diluent to reduce the concentration of the product and match the osmotic pressure of the composition to that of the tissue fluids at the site of administration in the human body.

The disclosed composition accommodates a high concentration of acetaminophen of at least 225 mg per mL, which is significantly higher than the solubility of acetaminophen in water (ca. 13 mg per ml) at room temperature and, yet, can be refrigerated and diluted to lower concentrations with pharmaceutically acceptable diluents for the purpose of storage, shipping, co-administering the composition with various other therapeutic agents without causing precipitation or other undesirable physical and chemical changes to the API. This makes it possible to contain the maximum unit-dose in a volume that is significantly less than that of marketed products. The substantial reduction of product volume (e.g. from 100 ml in comparable products to approximately 5 ml in the concentrated product) offers a significant savings in shipping and storage costs.

Another advantage of the composition is in its ability to prevent chemical degradation of acetaminophen when exposed to dissolved oxygen or other oxidizing species without the addition of any antioxidants or chelating agents. Additionally, the chemical and physical stability of acetaminophen in the disclosed composition is not affected by the direct exposure to atmospheric oxygen during typical periods of time involved in the manufacture, storage and use of such pharmaceutical products. The solution composition does not require a low concentration of dissolved oxygen in the solution or in its surrounding environment, does not require the addition of buffers or controlled pH, does not require free-radical scavengers and antagonists, does require antioxidants, metal chelators (e.g. ethylenediamine tetra acetic acid (EDTA) or salts thereof) other organic acids or mannitol and, yet, remains physically and chemically stable for extended periods of time. As a further advantage, the concentrated solution of acetaminophenin PEG 300 can be thermally sterilized by the application of heat, rendering it suitable for parenteral administration.

In one embodiment, the composition comprises polyethylene glycol (PEG), acetaminophen and trace water such that there is at least 200 mg of acetaminophen per mL of PEG. In one embodiment, the average molecular weight of the PEG is between 200 and 400. In one embodiment, the PEG is PEG 200 with an average molecular weight of about 200. As used in this specification, the term “about” refers to a variation of 25% or less. In another embodiment, the PEG is PEG 300. In yet another embodiment, the PEG is PEG 400. Trace water refers to less than 10% (m/v) water. In one embodiment, there is less than 5% (m/v) water. In another embodiment, there is less than 1% (m/v) water. Additives (e.g. buffers, chelates, etc.) may be included in the composition but are not required to provide a stable solution. In one embodiment, the solution is at least 75% (m/m) polyethylene glycol. In one embodiment, the composition consists essentially of PEG, acetaminophen and trace water such that there is at least 200 mg of acetaminophen per mL of PEG. In yet another embodiment, the composition consists of PEG, acetaminophen and trace water such that there is at least 200 mg of acetaminophen per mL of PEG.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A composition of matter comprising:
a polyethylene glycol in liquid form;
an acetaminophen dissolved in the polyethylene glycol, the acetaminophen being present in a concentration of at least 200 mg per mL of the polyethylene glycol; and
less than 10% (m/v) water.

2. The composition of matter as recited in claim 1, wherein the polyethylene glycol has an average molecular weight of between about 200 g per mole and about 400 g per mole.

3. The composition of matter as recited in claim 1, wherein the polyethylene glycol has an average molecular weight of about 200 g per mole.

4. The composition of matter as recited in claim 1, wherein the polyethylene glycol has an average molecular weight of about 300 g per mole.

5. The composition of matter as recited in claim 1, wherein the polyethylene glycol has an average molecular weight of about 400 g per mole.

6. The composition of matter as recited in claim 1, wherein the water is present in a concentration of 5% (m/v) or less.

7. The composition of matter as recited in claim 1, wherein the water is present in a concentration of 1% (m/v) or less.

8. The composition of matter as recited in claim 1, wherein the composition of matter is at least 75% (m/m) the polyethylene glycol.

9. A package comprising the composition of matter as recited in claim 1, the composition of matter being disposed within a container selected from the group consisting of a syringe and a flexible bag.

10. A composition of matter consisting essentially of:
a polyethylene glycol in liquid form;
an acetaminophen dissolved in the polyethylene glycol, the acetaminophen being present in a concentration of at least 200 mg per mL of the polyethylene glycol; and
less than 10% (m/v) water.

11. The composition of matter as recited in claim 10, wherein the polyethylene glycol has an average molecular weight of between about 200 g per mole and about 400 g per mole.

12. The composition of matter as recited in claim 10, wherein the polyethylene glycol has an average molecular weight of about 200 g per mole.

13. The composition of matter as recited in claim 10, wherein the polyethylene glycol has an average molecular weight of about 300 g per mole.

14. The composition of matter as recited in claim 10, wherein the polyethylene glycol has an average molecular weight of about 400 g per mole.

15. The composition of matter as recited in claim 10, wherein the water is present in a concentration of 5% (m/v) or less.

16. The composition of matter as recited in claim 10, wherein the water is present in a concentration of 1% (m/v) or less.

17. The composition of matter as recited in claim 10, wherein the composition of matter is at least 75% (m/m) the polyethylene glycol.

18. A package comprising the composition of matter as recited in claim 10, the composition of matter being disposed within a container selected from the group consisting of a syringe and a flexible bag.

19. A composition of matter consisting of:
a polyethylene glycol in liquid form, the polyethylene glycol having an average molecular weight of about 300 g per mole;
an acetaminophen dissolved in the polyethylene glycol, the acetaminophen being present in a concentration of at least 200 mg per mL of the polyethylene glycol; and
less than 5% (m/v) water.

20. A package comprising the composition of matter as recited in claim 19, the composition of matter being disposed within a container selected from the group consisting of a syringe and a flexible bag.
